SEQUENCE LISTING (15 sequences)

SEQ ID NO: 1 - protein, 8 aa, Human immunodeficiency virus
RTLNAWVK

SEQ ID NO: 2 - protein, 9 aa, Human immunodeficiency virus
VGGHQAAMQ

SEQ ID NO: 3 - protein, 6 aa, Human immunodeficiency virus
WDRLHP

SEQ ID NO: 4 - protein, 8 aa, Human immunodeficiency virus
PRGSDIAG

SEQ ID NO: 5 - protein, 6 aa, Human immunodeficiency virus
GLNKIV

SEQ ID NO: 6 - protein, 6 aa, Human immunodeficiency virus
VRMYSP

SEQ ID NO: 7 - protein, 5 aa, Human immunodeficiency virus
QGPKE

SEQ ID NO: 8 - protein, 8 aa, Human immunodeficiency virus
FRDYVDRF

SEQ ID NO: 9 - protein, 5 aa, Human immunodeficiency virus
LRAEQ

SEQ ID NO: 10 - protein, 7 aa, Human immunodeficiency virus
WMTETLL

SEQ ID NO: 11 - protein, 7 aa, Human immunodeficiency virus
WMTDTLL

SEQ ID NO: 12 - protein, 7 aa, Human immunodeficiency virus
QNANPDC

SEQ ID NO: 13 - protein, 7 aa, Human immunodeficiency virus
EEMMTAC

SEQ ID NO: 14 - protein, 8 aa, Human immunodeficiency virus
ACQGVGGP

SEQ ID NO: 15 - protein, 231 aa, Human immunodeficiency virus
PIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCETILKALGPGATLEEMMTACQGVGGPGHKARVL

(19) United States
(12) Patent Application Publication
(10) Pub. No.: US 2010/0048407 A1
(43) Pub. Date: Feb. 25, 2010

(54) METHOD FOR DETECTION OF HUMAN IMMUNODEFICIENCY VIRUS

(75) Inventor: Kalle Saksela, Helsinki (FI)

Correspondence Address: BIRCH STEWART KOLASCH & BIRCH, PO BOX 747, FALLS CHURCH, VA, 22040-0747, US

(73) Assignee: Next Biomed Technologies NBT Oy, Helsinki (FI)

(21) Appl. No.: 12/522,838

(22) PCT Filed: Jan. 17, 2008
(86) PCT No.: PCT/FI08/50012

§ 371 Date: Oct. 1, 2009

Related U.S. Application Data
(60) Provisional application No. 60/885,352, filed on Jan. 17, 2007.

(30) Foreign Application Priority Data
Jan. 17, 2007 (FI) 20075028

(51) Int. Cl. C40B 10/00; G01N 33/53; C07K 2/00

(52) U.S. Cl. 506/1; 436/501; 435/7.1; 530/300

(57) ABSTRACT

The present invention provides bioengineered high affinity polypeptides for use in a method for the detection of the presence of human immunodeficiency virus, HIV, in a biological sample. The present invention also provides a method for producing bioengineered high affinity polypeptides.

### FIELD OF THE INVENTION

The invention relates to the diagnosis and clinical management of human immunodefiency virus infections.

### BACKGROUND OF THE INVENTION

Despite the fact that human immunodefiency viruses do not contain antibody epitopes (i.e. peptide stretches with sufficient length and immunogenicity) that would be conserved enough to allow reliable and quantitative antibody-mediated detection, diagnostic tests that involve immunological detection of human immunodefiency virus HIV-1/2 capsid proteins are in clinical use. The problem of the prior art is that all globally circulating virus strains as well as quasispecies within a single infected individual are not detected. Another problem is that the current immunoassays cannot detect all viruses with same affinity in a way that the binding signal obtained would be directly proportional to the abundance of the virus regardless of its origin. Moreover, the binding affinity of traditional antibodies (see, e.g., U.S. Pat. No. 6,432,633) used for detection of HIV antigens is not high enough to allow development of a sufficiently sensitive assay that would be useful in diagnosing HIV infection or for monitoring viral load during follow up of the antiretroviral therapy of HIV-infected individuals. Although detection of HIV antigens could in theory be a superior approach to these diagnostic needs, because of the limitations discussed above, today PCR-based methods or serology (alone or in combination with the currently available suboptimal antigen detection technology) are used for these applications. The invention described here offers a solution to the limitations in diagnostic detection of virion associated HIV proteins that are inherent to the currently used immunological methods.

Schupbach et al. (Journal of Medical Virology, 2001, 65:225-232) discloses that heat-denatured, amplification-boosted p24 antigen can be used as an alternative to HIV RNA testing in order to monitor the treatment of HIV infection. Respess et al. (Journal of Clinical Microbiology, 2005, 43(1):506-508) and Knuchel et al. (Journal of Clinical Virology, 2006, 36:64-67) also disclose ultrasensitive p24 antigen assays as an alternative to HIV RNA testing.

Boder et al. (PNAS, 2000, 97(20):10701-10705) discloses directed evolution of antibody fragments with monovalent femtomolar antigen-binding affinity. Holliger and Hudson (Nature Biotechnology, 2005, 23(9):1126-1136) reviews engineered antibody fragments. Nygren and Uhlen (Current Opinion in Structural Biology, 1997, 7:463-469) and Hosse et al. (Protein Science, 2006, 15:14-27) review engineering of protein display scaffolds for molecular recognition.

Binz et al. (Nature Biotechnology, 2005, 23(10):1257-1268) and Hey et al. (Trends in Biotechnology, 2005, 23(10):514-422) review engineering of novel binding proteins from nonimmunoglobulin domains.

However, none of the above-mentioned prior art publications or combinations thereof disclose bioengineered high affinity polypeptides designed to bind at least two or three amino acid residues long conserved epitopes of the p24 antigen, the production of said polypeptides and the use of said polypeptides in an HIV assay provided by the present invention.

### BRIEF DESCRIPTION OF THE DRAWING

FIG. 1. Amino acid sequence of p24 protein of a representative HIV-1 strain. The FIGURE shows relative conservation of the residues of p24 among clades A-K and various circulating recombinant viruses of the predominant M-type of HIV-1 as well as O- and N-type viruses and related SIV viruses from chimpanzees. Score of 1 indicates conservation of more than 99.75%, score of 2 indicates conservation of >99.50%, score of 3 indicates conservation of >99.00%, score of 4 indicates conservation of >98.00%, and score of 5 indicates conservation of >97.00% (the score is shown above each residue). X indicates that presence of two alternative residues is >99.75% conserved in this position. Residues that are less than 97% conserved are not scored. Residues with a score of 1 or 2 are indicted in boldface. Potential BHAP targets are underlined. Note that the side chains of all the amino acid in the underlined peptide regions may not contribute equally or at all to BHAP recognition. Thus, the recognition motif of a given BHAP could for example be WDRxHP.

### DETAILED DESCRIPTION OF THE INVENTION

The following definitions are provided for some terms used in this specification.

“Antibody” in its various grammatical forms is used herein as a collective noun that refers to a population of immunoglobulin molecules and/or immunologically active portions of immunoglobulin molecules, i.e., molecules that contain an antigen binding site or a paratope.

An “antigen-binding site”, a “paratope”, is the structural portion of an antibody molecule that specifically binds an antigen.

“Single-chain antibody” (scFv) is used to define a molecule in which the variable domains of the heavy and light chain of an antibody are joined together via a linker peptide to form a continuous amino acid chain synthesised from a single mRNA molecule (transcript).

“Immunoassay” is a biochemical test that measures the level of a substance in a biological liquid, typically serum, plasma, urine, or other body fluids, using the reaction of an antibody or antibodies to its antigen. The assay uses the specific binding of an antibody to its antigen. Monoclonal antibodies are often used because they usually bind to a single site of a molecule to be detected, and therefore provide more specific and accurate testing, which is not interfered by other molecules in the sample. The antibodies used must have a high affinity for the antigen. The presence of the antigen can be measured for instance in the diagnosis of infectious diseases by detecting the microbe specific molecular structures. Detecting the quantity of the antigen can be achieved by a variety of methods. One of the most common used techniques is to label the antigen or antibody. The label may consist of an enzyme (Enzyme ImmunoAssay, EIA), fluorescence (FIA), luminescence (LIA) or they can be based on agglutination, nephelometry, turbidimetry or immunoblotting (Western Blot).

Immunoassays can be either competitive or non-competitive, and they can be homogeneous or heterogeneous. In a competitive assay, the antigen in the sample competes with the labelled antigen to bind with antibodies. The amount of labelled antigen bound to the antibody site is then measured. The response will be inversely proportional to the concentration of antigen in the sample, because the greater the response, the less antigen in the sample is available to compete with the labelled antigen.

In non-competitive immunoassays, often referred to as “sandwich assay”, antigen in the sample is bound to the “capture” antibody and the amount of the labelled antibody on the site is measured. Unlike in the case of competitive assay the result will be directly proportional to the concentration of the antigen.

A heterogeneous immunoassay will require an extra step to remove unbound antibody or antigen from the site, usually using a solid phase material. Homogenous assays do not require the separation phase to remove the unbound antibody or antigen molecules. Immunoassays have a particularly important role in the diagnosis of HIV.

The abbreviation “BHAP” refers to “a bioengineered high affinity polypeptide”, which is a molecule that has been generated and optimized using recombinant DNA methodologies, and has capacity to bind to a ligand. For example, single-chain antibodies and their derivatives can serve as BHAPs.

The abbreviation “COPOS” refers to “conserved polypeptide structure”, which is a structure typically formed by two or more amino acid residues that tend to be constant even in otherwise highly variable proteins, such as many viral proteins, and can serve as a ligand for a BHAP. COPOS may overlap with an antigenic epitope, but may not be targeted by a traditional antibody.

As used herein, the term “specifically binding”, or “specifically recognizing”, or the expression “having binding specificity to an epitope” refers to a low background and high affinity binding between a BHAP or a fragment or derivative thereof and its target molecule (i.e. lack of non-specific binding). In other words, the terms (and equivalent phrases) refer to the ability of a binding moiety (e.g., a receptor, antibody, ligand or antiligand) to bind preferentially to a particular target molecule (e.g., ligand or antigen) in the presence of a heterogeneous population of proteins and other biologics (i.e., without significant binding to other components present in a test sample). Typically, specific binding between two entities, such as a ligand and a receptor, means a binding affinity of at least about 10⁶ M⁻¹, and preferably at least about 10⁷, 10⁸, 10⁹, or 10¹⁰ M⁻¹, more preferably at least about 10¹¹, 10¹², 10¹³, 10¹⁴ or 10¹⁵ M⁻¹.

The terms “biopanning” and “phage display library” are used herein in the same way as in the US Patent Application No. 2005/0074747 (Arap et al.).

Further, the classic definition of an antigen is “any foreign substance” that elicits an immune response (e.g., the production of specific antibody molecules) when introduced into the tissues of a susceptible animal and is capable of combining with the specific antibodies formed. Antigens are generally of high molecular weight and commonly are proteins or polysaccharides. Polypeptides, lipids, nucleic acids and many other materials can also function as antigens. Immune responses may also be generated against smaller substances, called haptens, if these are chemically coupled to a larger carrier protein, such as bovine serum albumin, keyhole limpet hemocyanin (KLH) or other synthetic matrices. A variety of molecules such as drugs, simple sugars, amino acids, small peptides, phospholipids, or triglycerides may function as haptens. Thus, given enough time, just about any foreign substance will be identified by the immune system and evoke specific antibody production. However, this specific immune response is highly variable and depends much in part on the size, structure and composition of antigens. Antigens that elicit strong immune responses are said to be strongly immunogenic.

Characteristics of a good antigen include:
Areas of structural stability and chemical complexity within the molecule.
Significant stretches lacking extensive repeating units.
A minimal molecular weight of 8,000-10,000 Daltons, although haptens with molecular weights as low as 200 Da have been used in the presence of a carrier protein.
The ability to be processed by the immune system.
Immunogenic regions which are accessible to the antibody-forming mechanism.
Structural elements that are sufficiently different from the host.
For peptide antigens, regions containing at least 30% of immunogenic amino acids: K, R, E, D, Q, N.
For peptide antigens, significant hydrophilic or charged residues.

In the case of detection of human immunodefiency virus, HIV, the problem is that the antigenic sites of the virus are constantly and rapidly changing. The solution of the present invention is to provide means to prepare a bioengineered high affinity polypeptide (BHAP), which specifically binds to at least two or three amino acid residues long epitopes of the p24 polypeptide, which would be difficult or impossible to detect with regular antibodies. The BHAPs thus obtained can be used in detection methods in the same way as antibodies and are thus useful in detecting the presence of human immunodeficiency virus in a biological sample.

The present invention provides a method for detecting the presence of human immunodeficiency virus in a biological sample, the method comprising

a) contacting said sample or a fraction thereof with a bioengineered high affinity polypeptide (BHAP) rationally targeted to bind to conserved structural determinants (COPOS) formed by the backbone and side chain atoms of at least two or three amino acid residues or more within short, typically less than ten residues, peptide regions in the p24 polypeptide of HIV.
b) detection of a complex of said bioengineered high affinity polypeptide and p24 or a fragment thereof, the presence of said complex indicating the presence of HIV in said sample.

The COPOS binding determinants are preferably located within the following conserved 5- to 9-mer peptides in the p24 polypeptide of HIV:

| | |
|---|---|
| R T L N A W V K, | (SEQ ID NO: 1) |
| V G G H Q A A M Q, | (SEQ ID NO: 2) |
| W D R L H P, | (SEQ ID NO: 3) |
| P R G S D I A G, | (SEQ ID NO: 4) |
| G L N K I V, | (SEQ ID NO: 5) |
| V R M Y S P, | (SEQ ID NO: 6) |
| Q G P K E, | (SEQ ID NO: 7) |
| F R D Y V D R F, | (SEQ ID NO: 8) |
| L R A E Q, | (SEQ ID NO: 9) |
| W M T E T L L, | (SEQ ID NO: 10) |
| W M T D T L L, | (SEQ ID NO: 11) |
| Q N A N P D C, | (SEQ ID NO: 12) |
| E E M M T A C, | (SEQ ID NO: 13) |
| and | |
| A C Q G V G G P. | (SEQ ID NO: 14) |

However, the invention is not limited only to these peptides above, because it is clear to a skilled person of the art, that other epitopes derived from p24 polypeptide of HIV and useful in this invention may be discovered by further computational analysis of known p24 sequences or sequences which are to be discovered. Computational sequence identity comparisons can be conducted using an amino acid or nucleotide sequence comparison algorithm such as those known to a skilled person of the art. For example, one can use the BLASTN algorithm.

Preferably, the COPOS binding determinant consists of 2 to 3, 2 to 4, 2 to 5, 2 to 6, 3 to 4, 3 to 5, 3 to 6, 2 to 7, or 3 to 7 adjacent or non-contiguous amino acid residues. More preferably the COPOS binding determinant consists of 2, 3, 4, 5, 6, or 7 adjacent or non-contiguous amino acid residues.

The biological sample to be tested is preferably a blood sample. Said sample or fraction thereof is preferably subjected to conditions that denature polypeptides in the sample before performing step a) of the method above.

Further, the present invention provides a method for producing a bioengineered high affinity polypeptide (BHAP) which is able to specifically bind to an at least two to three adjacent or non-contiguous amino acids long epitope in a conserved region of the p24 antigen of HIV, the method comprising the steps of:

a) selecting an at least two amino acid long conserved region in the p24 antigen by computational analysis of known amino acid sequences of the p24 antigen;
b) preparing a peptide based on the selected conserved region of the p24 antigen;
c) contacting a library of particles expressing binding proteins with said peptide, preferably said library is a phage library of single chain antibodies;
d) isolating those particles which express binding proteins having binding activity towards said peptide;
e) subjecting nucleic acid obtained or derived from the particle(s) isolated in step d) to mutagenesis;
f) preparing a library of particles expressing binding proteins based on the particles obtained from step e);
g) contacting a library obtained from step f) with said peptide or a fragment thereof;
h) isolating those particles which express binding proteins having improved binding activity towards said peptide or a fragment thereof; said improved binding activity may be, e.g., higher affinity or better specificity;
i) repeating steps e) to h) one or more times;
j) obtaining a bioengineered high affinity polypeptide which is able to specifically bind an at least two to three adjacent or non-contiguous amino acids long epitope in a conserved region of the p24 antigen of HIV from the particles obtained from step i).

The present invention also provides bioengineered high affinity polypeptides (BHAP) obtained by the method disclosed above.

The publications and other materials used herein to illuminate the background of the invention, and in particular, to provide additional details with respect to its practice, are incorporated herein by reference. The present invention is further described in the following examples, which are not intended to limit the scope of the invention.

### EXAMPLES

### Example 1

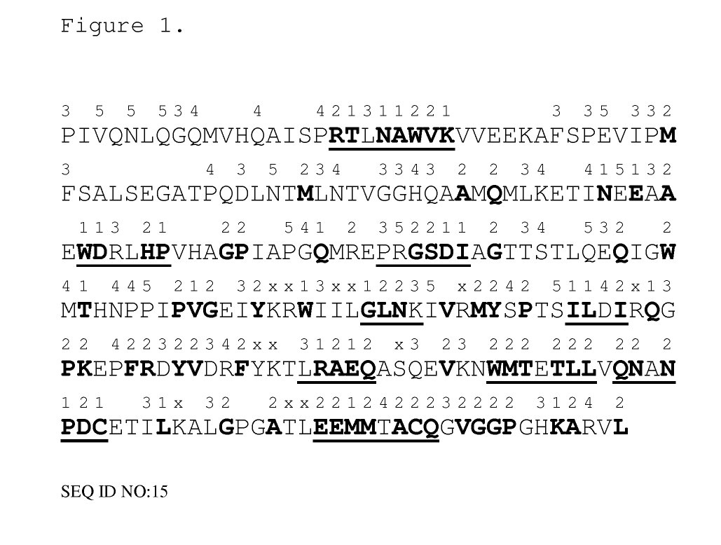

To identify COPOS determinants in HIV p24 a large number of individual amino acid sequence available in public databases, such as http://www.hiv.lanl.gov/content/index, were aligned with each other, and the relative conservation of each amino acid residue was evaluated. Based on this analysis peptides typically shorter than ten residues and containing at least two amino acids that are conserved in more than 99% of the sequences were selected for further analysis (see FIG. 1).

Following generation of potential BHAP molecules that bind to these peptides, for example by screening scFv phage libraries (basic principles of screening recombinant antibody libraries are reviewed by Hoogenboom, Nature Biotechnology 23(9): 1105-1116), the residues that account for this binding are identified using peptide array technology. BHAP recognition motifs that consist of highly conserved sets of HIV p24 residues are then considered as COPOS determinants. Such sets consist typically of two to five residues, which may or may not be positioned immediate adjacent to each other in the HIV 24 polypeptide chain. Thus, any combination of two or more residues within the peptide sequences listed above (SEQ ID NOS: 1-14) is a potential COPOS to be used in detection of HIV p24.

### Example 2

Synthetic peptides containing one or several potential COPOS determinants are used to screen large libraries of polypeptides that can serve as BHAP precursors using affinity based selection methods. For example, the ETH-2-Gold phage display library generated by Neri and colleagues (Proteomics 5:2340-2350, 2005) containing three billion individual recombinant antibody clones is screened for polypeptides that can specifically interact with COPOS-containing peptides. Several libraries containing potential ligand binding polypeptides based on non-Ig-derived polypeptides also exist (see e.g. Nature Biotechnology 23:1257-1268, 2005) or can be designed de novo, and are used to screen for polypeptides as to develop BHAPs. In addition to screening of such BHAP precursor libraries with synthetic peptides, recombinant proteins containing one or more potential COPOS determinants, as well as denatured HIV capsid proteins (p24) are used as ligands in affinity selection.

BHAP precursors that bind both to denatured p24 as well as a defined COPOS-containing peptide are chosen for further development. Initially, the detailed binding determinants in their COPOS-containing target peptides are elucidated using peptide array technology, such as PepSpot™ peptide membranes developed by JPT Peptide Technologies GmbH, and BHAP precursors that bind to maximally conserved structures in these peptides (=bona fide COPOS elements) are selected for improvement via bioengineering. Binding affinity of these pre-BHAPs is maximized, and if necessary their binding specificity further biased to maximally conserved molecular determinants in p24 (see FIG. 1) via reiterated mutagenesis and affinity selection, as described by the inventors in their previous studies related to SCA engineering (Biochemistry 41:12729-12738, 2003). Both random mutagenesis using error-prone PCR as described in Biochemistry article cited above or other similar techniques, as well as targeted mutagenesis of the binding surfaces in the BHAPs, or combinations of these approaches are used. Traditional phage-display based on the M13-derived phagemid plus helper bacteriophage-mediated approach are used for affinity selection and amplification of the improved BHAP molecules, but other related screening methods can also be used.

The binding affinities and other salient properties are then characterized in detail. The properties of optimal BHAPs, which will then used as such or as various fusion protein derivatives for building of novel p24 detection assays include: 1) High affinity for heat-denatured HIV p24 protein, preferably meaning a dissociation constant lower than 10⁻¹² M, 2) absolute conservation of the cognate COPOS determinants in more than 99% of the relevant virus strains, and 3) good solubility and ease of large-scale recombinant production

1. Method for detecting the presence of human immunodeficiency virus, HIV, in a biological sample, the method comprising
a) contacting said sample or a fraction thereof with a bioengineered high affinity polypeptide (BHAP) rationally targeted to bind to conserved structural determinants (COPOS) formed by the backbone and side chain atoms of at least two or three amino acid residues or more within short, typically less than ten residues, peptide regions in p24 antigen; and
b) detecting a complex of said bioengineered high affinity polypeptide and p24 or a fragment thereof, the presence of said complex indicating the presence of HIV in said sample.

2. The method according to claim 1, wherein the COPOS binding determinants are located within the following conserved 5- to 9-mer peptides in the p24 antigen of HIV:

| | |
|---|---|
| R T L N A W V K, | (SEQ ID NO: 1) |
| V G G H Q A A M Q, | (SEQ ID NO: 2) |
| W D R L H P, | (SEQ ID NO: 3) |
| P R G S D I A G, | (SEQ ID NO: 4) |
| G L N K I V, | (SEQ ID NO: 5) |
| V R M Y S P, | (SEQ ID NO: 6) |
| Q G P K E, | (SEQ ID NO: 7) |
| F R D Y V D R F, | (SEQ ID NO: 8) |
| L R A E Q, | (SEQ ID NO: 9) |
| W M T E T L L, | (SEQ ID NO: 10) |
| W M T D T L L, | (SEQ ID NO: 11) |
| Q N A N P D C, | (SEQ ID NO: 12) |
| E E M M T A C, | (SEQ ID NO: 13) |
| and | |
| A C Q G V G G P. | (SEQ ID NO: 14) |

3. The method according to claim 1, wherein the COPOS binding determinant consists of 2 to 3, 2 to 4, 2 to 5, 2 to 6, 3 to 4, 3 to 5, 3 to 6, 2 to 7, or 3 to 7 adjacent or non-contiguous amino acid residues.

4. The method according to claim 3, wherein the COPOS binding determinant consists of 2, 3, 4, 5, 6, or 7 adjacent or non-contiguous amino acid residues.

5. The method according to claim 1, wherein the polypeptides of the sample or fraction thereof are denatured before performing step a).

6. The method according to claim 1, wherein said bioengineered high affinity polypeptide has affinity of 10⁻¹⁰ to 10⁻¹⁵ M to the epitope.

7. The method according to claim 1, wherein said bioengineered high affinity polypeptide is a single chain antibody or a derivative thereof.

8. The method according to claim 1, wherein said bioengineered high affinity polypeptide is a scFV or a derivative thereof.

9. The method according to claim 1, wherein said bioengineered high affinity polypeptide is obtained by subjecting a binding polypeptide to successive rounds of biopanning.

10. The method according to claim 9, wherein said biopanning is based on phage display systems.

11. The method according to claim 1, wherein the epitope is not immunogenic.

12. The method according to claim 1, wherein said sample is a blood sample.

13. The method according to claim 1, wherein said bioengineered high affinity polypeptide is labelled.

14. Method for producing a bioengineered high affinity polypeptide which is able to specifically bind to an at least two to three adjacent or non-contiguous amino acids long epitope in a conserved region of the p24 antigen of HIV, the method comprising the steps of:
a) selecting an at least two amino acid long conserved region in the p24 antigen by computational analysis of known amino acid sequences of the p24 antigen;
b) preparing a peptide based on the selected conserved region of the p24 antigen;
c) contacting a library of particles expressing binding proteins with said peptide;
d) isolating those particles which express binding proteins having binding activity towards said peptide;
e) subjecting nucleic acid obtained or derived from the particle(s) isolated in step d) to mutagenesis;
f) preparing a library of particles expressing binding proteins based on the particles obtained from step e);
g) contacting a library obtained from step f) with said peptide or a fragment thereof;
h) isolating those particles which express binding proteins having improved binding activity towards said peptide or a fragment thereof;
i) repeating steps e) to h) one or more times;
j) obtaining a bioengineered high affinity polypeptide which is able to specifically bind an at least two to three adjacent or non-contiguous amino acids long epitope in a conserved region of the p24 antigen of HIV from the particles obtained from step i).

15. The method according to claim 14, wherein said library is a phage library of single chain antibodies.

16. The method according to claim 14, wherein said bioengineered high affinity polypeptide has affinity of 10⁻¹² to 10⁻¹⁵ M to the epitope.

17. The method according to claim 14, wherein said peptide is selected from the group consisting of:

| | |
|---|---|
| R T L N A W V K, | (SEQ ID NO: 1) |
| V G G H Q A A M Q, | (SEQ ID NO: 2) |
| W D R L H P, | (SEQ ID NO: 3) |
| P R G S D I A G, | (SEQ ID NO: 4) |
| G L N K I V, | (SEQ ID NO: 5) |
| V R M Y S P, | (SEQ ID NO: 6) |
| Q G P K E, | (SEQ ID NO: 7) |
| F R D Y V D R F, | (SEQ ID NO: 8) |
| L R A E Q, | (SEQ ID NO: 9) |
| W M T E T L L, | (SEQ ID NO: 10) |
| W M T D T L L, | (SEQ ID NO: 11) |
| Q N A N P D C, | (SEQ ID NO: 12) |
| E E M M T A C, | (SEQ ID NO: 13) |
| and | |
| A C Q G V G G P. | (SEQ ID NO: 14) |

18. The method according to claim 17, wherein the epitope consists of 2 to 3, 2 to 4, 2 to 5, to 6, 2 to 7, 3 to 4, 3 to 5, 3 to 6 or 3 to 7 adjacent or non-contiguous amino acid residues.

19. The method according to claim 17, wherein the epitope consists of 2, 3, 4, 5, 6, 7 adjacent or non-contiguous amino acid residues.

20. A bioengineered high affinity polypeptides (BHAP) obtained by the method according to claim 14.

21. Use of the bioengineered high affinity polypeptides (BHAP) according to claim 20 for the detection of the p24 antigen of HIV in a biological sample.
